(19) United States
(12) Patent Application Publication
(10) Pub. No.: US 2012/0082626 A1
(43) Pub. Date: Apr. 5, 2012

(54) USE OF R-BAMBUTEROL AS INHALED MEDICAMENT AND COMBINATION THERAPIES FOR TREATMENT OF RESPIRATORY DISORDERS

(76) Inventor: Wen Tan, Corpus Christi, TX (US)

(21) Appl. No.: 13/376,272

(22) PCT Filed: Jun. 11, 2010
(86) PCT No.: PCT/US2010/001691

§ 371 Date: Dec. 5, 2011

Related U.S. Application Data
(60) Provisional application No. 61/268,778, filed on Jun. 16, 2009.

(51) Int. Cl. A61K 31/27; A61K 31/58; A61K 31/573; A61K 31/56; A61K 31/46; A61K 31/4409; A61K 31/522; A61K 31/4458; A61K 9/12; A61P 11/00; A61P 11/06; A61P 3/04; A61P 3/06; A61P 3/10; A61M 15/00; C07C 271/44

(52) U.S. Cl. 424/45; 514/483; 560/164; 514/171; 514/304; 514/291; 514/278; 514/357; 514/479; 514/263.35; 514/317; 128/203.12

(57) ABSTRACT

The present invention concerns with a new use of R-Bambuterol or Bambuterol as inhaled medicament for treatment of asthma, COPD and other respiratory disorders, and a new use of R-bambuterol or bambuterol and corticosteroids or other therapeutically active medicament as combined inhaled therapies. The invention also related to a new use of R-bambuterol with reduced drug tollerance and risk of exerbation of asthma associated with bambuterol in treatment of respritroy discorders.

### FIELD OF INVENTION

The invention related to the use of R-Bambuterol or Bambuterol as inhaled medicament for treatment of asthma and COPD and other respiratory disorders.

Inhalation of bambuterol aerosols into bronchioles and lungs can significantly improve the control of asthma or COPD with higher efficiency, faster onset and longer duration of action as well reduced toxicity comparing oral administration. The invention also related to use of R-bambuterol or bambuterol and corticosteroids or other therapeutically active medicament as combined inhaled therapies. In addition, use of R-bambuterol as inhaled medicament has more advantages over bambuterol. This invention also concern with a new use of R-bambuterol as medicament for treatment of respiratory disorders with reduced drug tollerance and risk of asthma excerbation associated with bambuterol.

### BACKGROUND OF INVENTION

Asthma and COPD are most common diseases. Bambuterol as a once daily oral dosage has been used for treatment of these diseases for almost 20 years. Bambuterol is a β2 agonist and a bronchospasmolytic agent. Bambuterol is also a chiral drug consisted of equal amount of R- and S enantiomer. R-bambuterol is the eutiomer and active in bronchial dilation, while S-bambuterol is the distiomer and inactive. S-bambuterol is also more cardiac toxic than R-bambuterol (Tan & Cheng, U.S. Patent. 2002). Bambuterol is a prodrug of terbutaline. Bambuterol is hydrolyzed by acetylcholinesterase (AChE) and butyrylcholinesterase (BuChE), into terbutaline, a known bronchial dilator after oral absorption. Bambuterol per se is also a potent inhibitor of the same enzymes. For this reason, the release of terbutaline is a slow process. After an oral administration of bambuterol, the blood concentration of active parent drug are slowly increased and has a duration up to 24 hours. Bambuterol therefore has a long bronchiospasmolytic action. Both R-bambuterol and bambuterol have relatively higher oral bio-availability (around 60-70%), and the time of maximum concentration (tmax) is around one hour.

Bambuterol as a prodrug was designed as oral dosage, it can be uptake by the lungs after oral administration due to its lipid phallic property and first pass protection in liver, its concentration in lungs is almost 20 times higher than that in plasma (Svensson, New drugs for Asthma Therapy, 1991) Therefore bambuterol can selectively target the lungs when administered orally.

However, there are disadvantages for the oral dosage of R-bambuterol or bambuterol. First, a slow onset of action. The time of maximum plasma concentration (tmax) of the parent drug, terbutaline, released from bambuterol after oral administration is four hours later than the tmax of terbutaline taken orally. (Olsson et. al., p. 36, table 1, U.S. patent, 1984). Bambuterol is therefore not suitable for fast control of asthmatic symptoms. Secondly, higher dosage is need for achieving effective parent drugs concentration in blood. To reach the same concentration of terbutaline in blood would need 5 times more bambuterol than terbutaline given orally on molar basis. The adverse effects of bambuterol in clinic are dose related (Gunn et al., Eur J. Clin Pharmacol 48, 1995). Regular use of larger dose of β₂ agonist could also lead to desensitization of β₂ receptors, and resulting in drug tolerance which may be linked to worsen asthma control or fatal asthma attack.

These disadvantages of oral dosage bambuterol may be overcome by a pulmonary delivery. However, Olsson et al. (Pharmaceutical Research,1984 see p. 21, Col. 2, line 9) demonstrated that bambuterol is inactive and lack bronchospasmolytic effects when inhaled into the lungs in anesthetized guinea pigs during asthma evoked by intravenously administrated histamine. The lack of action was attributed to slow absorption of bambuterol when inhaled. (Svenssion et al, Pharmaceutical Research, 1988, see p. 154, Col 2, line 41).

There are other studies in prior art also unfavourite the use of bambuterol as inhaled medicament for treatment of asthma. First, bambuterol is a prodrug and released slowly in vitro and in vivo. Directly use of bambuterol on the bronchial and lung preparations in vitro showed no relaxation effects. (Olsson et al., U.S. patent, 1983). Secondly, Svensson et al. (Ryrfeldt et al 1988) has studied the lung uptake and transformation of H3-bambuterol using isolated perfused guinea pig lungs. The total amount of bambuterol uptake by the lung is only 1.31% (30.5±4.8 pmol/lung) when a clinic relevant amount of bambuterol was perfused. Only 0.4% of terbutaline, the parent drug can be detected (0.15 pmol/lung) from the total bambuterol perfused. (averaged according to table II, FIG. 4, p 154, of the cited reference by Ryrfeldt et al, 1988). This little terbutaline (parent drug) is obviously insufficient for any anti-asthma effects. An in vitro metabolic study showed that bambuterol, is first transformed into inactive mono-carbamate bambuterol and then gradually hydrolyzed into the active parent drug, terbutaline. The first step of transform will dominate and litter terbutaline was formed at the initial phase, while major terbutaline will be formed at a much later phase (Svenssion et al., 1988, see p. 3871, FIG. 5).

In prior art, it seems that bambuterol has a slow release nature, hardly to be absorbed via lungs and inactive when inhaled. The prior art seems to teach away a skilled artisan for developing an inhaled form of bambuterol for pulmonary delivery. In fact, bambuterol has always been used as oral dosage forms since it was marked 20 years ago. There is no reports on use of bambuterol as inhaled drug for treatment of respiratory disorders in prior art.

The inventor believes that inhaled R-bambuterol or bambuterol can be effectively uptake and reasonable amount of the active parent drug can be released locally in light of the following understandings and facts: 1, bambuterol can be nubelized or micronised and able to reach the small bronchioles and deep portion of the lungs after inhaled. 2, the blood-air interface consist of pulmonary cells, basement membrane and alveolar capillary should be permeable to the mironized bambuterol, and further the bambuterol should be readily hydrolyzed by AchE and BuchE within the lungs. 3, the phospholipid surfactants in the lung mucosa could be useful for inhaled bambuterol to facilitate the dispersion and penetration through the blood-air interphase of pulmonary alveolar.

In addition, the inventor notices that there were certain drawbacks and limitations in previous experiments noted above by Svenssion et al (1984 and 1988) since they were done in anesthetized and unsensitized animals or isolated lungs in vitro. The lack of uptake of bambuterol and little transformation of active parent drug, terbutaline, seen in Svenssion's experiments in isolated lungs may be due to several factors: 1, disformation and edema of blood-air interface in which the permeability is greatly impaired, 2, the lipid surfactants in lung were diluted and deficient in the perfusate. 3. decrease enzymatic activities of AchE in the lung tissue and lack of blood Bu-AchE in the perfusate. For these reasons, the inventor believes that Svenssion's studies may not rule out the possibilities that bambuterol can be readily absorbed and transformed into active parent drugs in therapeutically effective amount in conscious animals in vivo or in patients when administered by inhalation. 4, Olsson's (1984) experiments were conducted in anesthetized and unsensitized animals in instead of conscious and antigen sensitized animals. In addition, histamine was given intravenously instead of given antigen by inhalation. These differences may result in different outcomes in studying the anti-asthmatic effect of inhaled bambuterol. Svenssion's results may not reflect the situation in conscious animals or in antigen-sensitized animal which is more clinically relevant.

Current asthma guidelines recommend augmenting therapy with long-acting beta agonists in patients whose symptoms are not adequately controlled with an inhaled corticosteroid. However, clinic reports and recent clinic trials indicate the use of the LABA salmeterol or femoterol will increase the risk of asthma-related death, which causing the U.S. Food and Drug Administration to issue warnings about salmeterol and similar medications. Therefore, There is a great need for a safer β agonist as inhaled medicament, specially the long acting beta agoinst for treating respiratory disorders.

### DETAILS OF THE INVENTION

The invention provides a novel use of R-bambuterol or bambuterol as inhaled aerosols for the treatment of respiratory disorders. By inhalation into bronchioles or lungs, R-bambuterol or bambuterol has a greater efficiency and increased duration of bronchodilator action and a rapid onset of action. In the invention, conscious animal and antigen sensitized guinea-pigs were used for the studying of pulmonary delivery of R-bambuterol or bambuterol. The experiment protocol and animal models used in the invention differ than what in prior art.

In one embodiment the invention provides that inhalation of R-bambuterol or bambuterol in only micrograms results in significant anti-asthmatic effects, while the same drugs in milligrams were required to achieve similar effects if administered orally. The oral dosage of R-bambuterol or bambuterol required for producing maximum anti-asthmatic effects (i.e. complete protection against asthmatic attack by histamine.) were 4 mg/kg and 8 mg/kg respectively, while only 256 μg/kg of R-bambuterol and 512 μg/kg of bambuterol were required when administered by inhalation. The ratio between the oral dosage and inhaled dosage required for maximum anti-asthmatic effect is 16:1 for R-bambuterol and same for bambuterol. These potent anti-asthmatic effects are in contrary to Olsson's study (1984) in which inhaled bambuterol was inactive for anti-asthma.

The invention also reveals by surprise a fast onset of action after inhalation of R-bambuterol or bambuterol. In an embodiment of the invention, the ovalbumin (OVA) sensitized guinea-pigs were first challenged with nebulized antigen to evoke asthma, then the animals were allowed to inhale nebulized R-bambuterol or bambuterol. The asthmatic symptoms were immediately relieved within matter of tenths of seconds to a couple of minutes after inhalation. In a further embodiment of the invention, the sensitized guinea-pigs were first pretreated with nebulized R-bambuterol or bambuterol by inhalation. After three minutes, the same animals were challenged with nebulized antigen. The inhalation of R-bambuterol or bambuterol produced a significant anti-asthma effects and a protection of pulmonary function during asthmatic attack as indicated by unchanged airway resistance and dynamic pulmonary compliance. These results of the invention indicate that R-bambuterol or bambuterol were rapidly uptake and transformed into active parent drugs after inhaled, which allow a fast relief of asthmatic symptoms. This result is in contrary to prior arts in which only traceable amount of terbutaline were transformed when bambuterol were perfused locally into the lungs (Svenssion, 1988.) and also contrary to Ossonly's results that bambuterol is inactive in protection against asthma due to the nature of slow release of active parent drug (Olsson et al., 1984). The fast onset of action in the invention can also not be explained by Svenssion's in vitro metabolic study noted above that the dominate product is mono-carbamate bambuterol and litter terbutaline at initial phase., while the major amount of terbutaline will be formed at much later phase. (Svensson et al., 1988).

Taking together of the prior art findings, the fast onset of action by inhaled bambuterol revealed in the invention can not be anticipated by a skilled person in art.

In another embodiment, the time course of action of R-bambuterol or bambuterol both by inhalation or oral were studied. It reveals that the R-bambuterol or bambuterol produced a maximum anti-asthmatic effect within 60 minutes (Tmax.ef) after inhalation, which is 4 hours later than the same drugs given orally. (Tmax.ef is 240 min in oral group). However there was no earlier decline of the maximum anti-asthma effects in inhaled group in comparing of oral group within 720 min or longer. This reveals that the duration of maxium effects were longer for inhalation, which indicates a higher afficacy for inhaled R-bambuterol or bambuterol. It provides a better protection against asthmatic attack than oral administraion. This advantage of higher efficacy of R-bambuterol or bambuterol when given by inhalation over by oral was unexpected from prior arts.

The invention further provides that there may be different metabolic mechanisms for inhaled R-bambuterol or bambuterol and for oral administration of the same drugs. As noticed above, a fast onset of action indicates the inhaled bambuterol in the lung were rapidly absorbed and transformed into its metabolites and active parent drugs. These in turn would speed-up the clearance of the drugs inhaled into the lungs, therefore may shorten the duration of its action. However, the invention reveals that the maximum anti-asthmatic effects of R-bambuterol and bambuterol by inhalation come 4 hours faster than did by oral administration, however, in the invention the duration of the effects can still last up tof 24 hour as did by oral administration, Therefore, the total duration for maximum action or effects is longer for inhalation than what for oral administration of the same drugs. These descripencies clearly indicate a different phamcokinetic profile. This is not known in prior art.

In one aspect, the invention reveals that there is greatly improved control of asthma via pulmonary delivery of R-bambuterol or bambuterol by inhalation at a much lower doses. Reduced dose not only can reduce adverse effects associated with the drug per se but also reduce the risk of drug tolerance due to desensitization, which may result in worsening controlled asthma and fatal or non-fatal asthma attack.

In an embodiment the invention also reveals for the first time that pretreatment of R-bambuterol by inhalation provided a fully protection against the asthma attack by antigen challenge, On the other hand, inhalation of S-bambuterol provided no such protection at all. The invention further reveals that the anti-asthmatic effects of inhaled racemic bambuterol resides within the R-enantiomer. The S-enantiomer of bambuterol is inactive in this regard.

In another embodiment the invention reveals for the first time that inhalation of R-bambuterol can improve pulmonary function by reduced airway resistance and enhanced pulmonary dynamic compliance in OVA sensitized guinea pigs at resting state while the asthma was not evoked. However, inhalation of S-bambuterol has the opposite effects which worsens the pulmonary function by increasing airway resistance and reducing pulmonary dynamic compliance at resting state. In addition, oral administration of S-bambuterol can significantly enhance the asthmatic response to OVA challenge than control. These effects of S-Bambuterol at both resting and asthma states may be related to the exacerbation of asthma or hyper-responsiveness of airways associated with the use of β2 agonists including bambuterol found in clinic practise. Therefore, R-bambuterol is a better alternative to be used as inhaled or oral drug over bambuterol to avoid the risk of asthma excerbation related with use of bambuterol. This excerbation of asthma by S-bambuterol were demonstrated directly for the first time by this invention. It can not be anticipated in prior arts.

In an additional embodiment, Animals were pretreated with R-bambuterol or S-babmuterol seperatedly for period of two weeks to induce a drug tolerance. The anti-asthmas effects of R-bambuterol were then evaluated by measuring the changes in Airway resistance (Raw) and dynamic pulmonary compliance (Cdyn). It reveals, for the first time, that the protection effects of R-bambuterol against asthamatic attack were significantly reduced in S-bambuterol pretreated group comparing to control, indicating a drug tolerance. Furthermore, this reduction was significant more than R-bambuterol pretreated group. This indicates that frequent use of racemic bambuterol (containing 50% S-bambuterol) is more likely to develop drug tolerance toward R-bambuterol or racemic bambuterol (Since R-bambuterol is the active ingredient of racemic bambuterol) than frequent use of R-bambuterol. Therefore, for reducing drug tolerance, R-babmtuerol is a better alternative for use in protection of asthma than racemic bambuterol. This result can not be anticipated from prior arts.

The said R-enantiomer bambuterol should be substantially optic pure. It should has an enantiomer excess value between 90%-99%. The amount of S-enantiomer bambuterol should be no more than 5% by weight, preferably the R-bambuterol should be free of S-enantiomer and the enantiomer excess value should be no less than 99%.

Suitable daily doses of R-bambuterol as inhaled drugs into bronchioles and lungs for treatment of asthma and COPD may be, for example in the range of 0.02 to 2.0 mg, a preferable dose in range 60 to 250 μg; per actuation or per treatment may be, for example in the range 20 to 250 μg of R-bambuterol; Suitable daily doses of bambuterol as inhaled drugs may be, for example in the range of 0.04 to 4.0 mg, a preferable dose in the range 125 to 500 μg; per actuation or per treatment may be, for example in the range 40 to 500 μg of bambuterol. The administration of either R-bambuterol or bambuterol may be, for example from 1-8 times a day, and giving for example 1,2,3,4 actuation or puffs each time. For children the doses may be further reduced. Since different inhaled formulations and inhalation devices may require different amount of the active ingredients for thrapeutical effective amount of deliver of R-bambuterol or bambuterol into the bronchioles and lungs, the actual dosage of drugs used in the preparation of different inhaled formulation may be adjusted. In addition, the suitable inhaled doses of R-bambuterol or bambuterol for patients can be determined by converting the doses in guinea-pigs into doses for human according to the weight or surface area, or by a proportion of their oral doses. The dose may be adjusted depending on the therapeutic objective of the use of the active agents and the age and condition of the patient.

In an embodiment of the invention, the inhalation form of R-bambuterol or bambuterol or their pharmaceutical acceptable salts may be, for example, an atomizable composition such as an aerosol comprising R-bambuterol or bambuterol in solution or dispersion in a propellant, or a nebulizable composition comprising a dispersion of the above active ingredient in an aqueous, organic or aqueous/organic medium. Suitable propellants include hydrocarbons particularly 1,1,1,2-tetrafluoroethane (HFA134a) and 1,1,1,2,3,3,3-heptafluoropropane (HFA227) or mixtures of two. Where the active ingredient, R-bambuterol or bambuterol or their pharmaceutical acceptable salts is present in suspension in the propellant, i.e. where it is present in particulate form dispersed in the propellant, the aerosol composition may also contain a lubricant and a surfactant, which may be chosen from those lubricants and surfactants known in the art. Other suitable aerosol compositions include surfactant-free or substantially surfactant-free aerosol compositions. The aerosol composition may contain up to about 5% by weight, for example 0.002 to 5%, 0.01 to 3%, 0.015 to 2%, 0.1 to 2%, 0.5 to 2% or 0.5 to 1%, by weight of the active ingredient, R-Bambuterol or bambuterol, based on the weight of the propellant. Where present, the lubricant and surfactant may be in an amount up to 5% and 0.5% respectively by weight of the aerosol composition. The aerosol composition may also contain a co-solvent such as ethanol in an amount up to 30% by weight of the composition, particularly for administration from a pressurized metered dose inhalation device. In another embodiment of the invention, the inhalable form is a dry powder, i.e. R-bambuterol or bambuterol or their pharmaceutical acceptable salts in a dry powder comprising optionally together with materials known as carriers in dry powder inhalation compositions, for example saccharides, including monosaccharides, disaccharides, polysaccharides and sugar alcohols such as arabinose, glucose, fructose, ribose, mannose, sucrose, trehalose, lactose, maltose, starches, dextran or mannitol. An especially preferred carrier is lactose. The dry powder may be in capsules of gelatin or plastic, or in blisters, for use in a dry powder inhalation device, Alternatively, the dry powder may be contained as a reservoir in a multi-dose dry powder inhalation device. The active ingredient may be a combination of R-bambuterol (or bambuterol) with an other medicament such as corticosteroid, anticholinergic etc. The inhalable form for combined medicament may be prepared similar as above in principle.

In another embodiment in the invention provides a novel pharmaceutical composition comprising effective amount of R-bambuterol or bambuterol or their salts and corticosteroids as a combined inhaled preparation for used in combination therapy for simultaneous, sequential or separate administration by inhalation into bronchial or lungs in the treatment of asthma or respiratory disorders. The use of this inhaled combination therapy of the invention will improve the therapeutic index and having an additive or synergistic effects. The corticosteroids include for example: budesonide, ciclesonide, beclomethasone, mometasone, flunisolide, fluticasone propionate, triamcinolone acetonide and/or its physiological accept sals and/or solvate thereof. The proportion of R-bambuterol with the corticosteroids may be, for example 1:1 to 1:60 on molar bases. A preferred ratio is 1:2 to 1:10; the more preferred ratio is 1:2 to 1:4; the proportion of bambuterol with above corticosteroids may be adjusted according to that the amount of bambuterol by weight should be twice as much as R-bambuterol.

The dose range of steroids may be adjusted depending on the therapeutic objective of the use of the active agents and the age and condition of the patient, for example, The range is 50 to 2000 μg of fluticasone propionate; 100-2000 μg of beclomethasone; and 50 to 4000 μg of budesonide etc. The inhaled formulation comprising R-bambuterol or bambuterol and one of the glucosteriod or corticosteriod in solution or dispersion in a propellant, or an inhalable nebulizable composition comprising a dispersion of medicaments in an aqueous, organic or aqueous/organic medium, or is mironised dry powder blended with lactose and packaged into a capsule for inhalers. The inhaled formulation can be prepared according to method mentioned above in. the invention.

In another embodiment in the invention provides an novel pharmaceutical composition comprising effective amount of R-bambuterol or bambuterol or their salts and short acting β2 agonists as a combined inhaled preparation for used in combination therapy for simultaneous, sequential or separate administration by inhalation into bronchial or lungs in the treatment of asthma or respiratory disorders. The use of this inhaled combination therapy of the invention will further improve the onset of action or having an additive or synergistic effects in the treatment of asthma, COPD and other respiratory disorders.

The short acting β2 agonists includes, for example: terbutaline, fenoterol, salbutamol, Orciprenaline, Clenbuterol; Clorprenaline, Reproterol, Bitolterol, Rimiterol etc. and their chiral eutimors. The inhaled formulation includes for example pressured metered dose inhaler, Insufflations or dry power inhaler, nebulizer etc.

The proportion of R-bambuterol with the short acting β2 agonists may be, for example 1:0.1 to 1:1 on molar bases. The proportion of bambuterol with short acting β agonists may be adjusted according to that the use of bambuterol should be twice as much as R-bambuterol. The inhaled formulation comprising R-bambuterol or bambuterol and one of the glucosteriod in solution or dispersion in a propellant, or an inhalable nebulizable composition comprising a dispersion of medicaments in an aqueous, organic or aqueous/organic medium, or is micronized dry powder blended with lactose and packaged into a capsule for inhalers. The inhaled formulation can be prepared according to method mentioned above in. the invention.

In an further embodiment in the invention provides a noval pharmceutical composition comprising effective amount of R-bambuterol or bambuterol or their salts and anticholinergics or muscurinic receptor antagonists as a combined inhaled preparation for used in combination therapy for simultaneous, sequential or separate administration by inhalation into bronchioles or lungs in the treatment of asthma or other respiratory disorders. The use of this inhaled combination therapy of the invention will activate the β2 receptors and inhibit the mscuranic receptors at the bronchioles at same time, therefore producing an additive or synergistic effects of bronchial dilation. The anticholinergics includes for example: ipratropium bromide, tiotropium, trospium, oxitropium, daratropium, atropine, homatropine, tropicamide, scopolamine, lycopyrolate, oxybutynin, tolterodine, and/or their salts. The proportion of the dosages of R-bambuterol and anticholinergics may be for example 1:0.1 to 1:2 on a molar basis. a preferred proportion may be for example 1:0.5. The proportion of bambuterol with anticholinergics may be adjusted according to that the amount of bambuterol should be twice as much as R-bambuterol. The dose range of anticholinergics may be adjusted depending on the therapeutic objective of the use of the active agents and the age and condition of the patient. The inhaled formulation comprising R-bambuterol or bambuterol and one of the anticholinergics in solution or dispersion in a propellant, or an inhalable nebulizable composition comprising a dispersion of medicament in an aqueous, organic or aqueous/organic medium, or is mironised dry powder blended with lactose and packaged into a capsule for inhalars. The inhaled formulation can be prepared according to method mentioned above in the invention.

In an further embodiment in the invention provides an novel pharmaceutical composition comprising effective amount of R-bambuterol or bambuterol or their salts and bronchodilators other than β2 agonist or anticholinergics for example: nitroxside, as a combined inhaled preparation for used in combination therapy for administration by inhalation into chronchioles or lungs in the treatment of respiratory disorders with improved therapeutic index or synergistic effects. The dose range of the above mentioned bronchial dilators may be adjusted depending on the therapeutic objective of the use of the active agents and the age and condition of the patient.

The inhaled formulation comprising R-bambuterol or bambuterol and for example nitroxside in solution or dispersion in a propellant, or an inhalable nebulizable composition comprising a dispersion of medicaments in an aqueous, organic or aqueous/organic medium, or is mironised dry powder blended with lactose and packaged into a capsule for inhalers. The inhaled formulation can be prepared according to method mentioned above in. the invention.

In an further embodiment in the invention provides an novel pharmaceutical composition comprising effective amount of R-bambuterol or bambuterol or their salts and anti-inflammatory or immunomodulatory agents include, for example: leukotriene receptor antagonists, interference and integrins, as a combined inhaled preparation for used in combination therapy for simultaneous, sequential or separate administration by inhalation into bronchioles or lungs in the treatment of respiratory disorders with improved therapeutic index or synergistic effects. The dose range of above anti-inflammatory agents may be adjusted depending on the therapeutic objective of the use of the active agents and the age and condition of the patient. The inhaled formulation comprising R-bambuterol or bambuterol and one of the medicament above in solution or dispersion in a propellant, or an inhalable nebulizable composition comprising a dispersion of medicament in an aqueous, organic or aqueous/organic medium, or is mironised dry powder blended with lactose and packaged into a capsule for inhalars. The inhaled formulation can be prepared according to method mentioned above in. the invention.

The invention provide additional novel uses of R-bambuterol or bambuterol as inhaled aerosol for lipid lowering, as an tocolytic agent for preterm labor, for treatment of spasm of gallbladder and for other symptoms or disorders which can be controlled via β2 receptor activation. A pulmonary delivery of R-bambuterol or bambuterol by aerosol inhalation in treatment of the above disorders will reduce adverse effects related to R-bambuterol or bambuterol. R-bambuterol which has further reduced adverse effects is preferred as an active ingredient in the above inhaled formulation. The dose range of R-bambuterol or bambuterol may be adjusted depending on the therapeutic objective of the use of the active agents and the age and condition of the patient. The inhaled formulation comprising R-bambuterol or bambuterol in solution or dispersion in a propellant, or an inhalable nebulizable composition comprising a dispersion of medicament in an aqueous, organic or aqueous/organic medium, or is mironised dry powder blended with lactose and packaged into a capsule for inhalers. The inhaled formulation can be prepared according to method mentioned above in the invention.

The pharmaceutical acceptable salts of R-bambuterol or bambuterol according to the invention include those formed with conventional pharmaceutical acceptable inorganic or organic acids for example: hydrochloride, hydrobromide, sulphate, hydrogen sulphate, dihydrogen phosphate, methanesulphonate, bromide, methyl sulphate, acetate, oxalate, maleate, fumarate, succinate, 2-naphthalene-sulphonate, glyconate, gluconate, citrate, tartaric, lactic, pyruvic isethionate, benzenesulphonate or para-toluenesulphonate.

### EXAMPLE 1

Protection against histamine-evoked asthma by inhaled R-bambuterol or bambuterol in conscious guinea pigs.

Test methods: Guinea pigs (Dunkin-Hartley strain, 200±30 g) were fasted overnight but given water ad libidum. The animals were restrained individually in a glass chamber and exposed to aerosol histamine generated by a nebulizer from a 0.2% aqueous solution of histamine under constant pressure at dose of 0.5 ml/min for a period of 15 sec. The animal was removed from the chamber, and its behavior was monitored. Assign of collapse and the latency time from exposure to collapse were recorded. Only the animals with latency time less than 120 sec, as an indication of sensitive to histamine, were chosen for later experiments. The animals chosen were allowed to recover completely by resting for 24 hours before experiments. R-bambuterol and racemic bambuterol hydrochloride were dissolved in saline and nublized in a nebulizer.

Study of dose-response: One hours before the exposure to aerosol histamine, animals were randomized into groups (n=8, equal sex), and Nublized R-bambuterol hydrochloride or bambuterol hydrochloride in doses of 63, 126, 252 and 504 μg/kg, and vehicle control were inhaled by the animals via a mouth and nasal mask. The numbers of collapsed animal as a result of asthmatic reaction to aerosol histamine were counted, and the latency times were recorded. Both of these parameters were used as quantitative measurements of the protective effects of the treatments on bronchospasm evoked by aerosol histamine. For animals showing no sign of severe asthma and which did not collapse over period of 360 sec, it counted as no collapse and the latency time was recorded as 360 sec.

Test results: The effects of inhaled R-bambuterol or bambuterol on histamine evoked asthma in conscious guinea-pigs are summarized in Tables 1-1 to 1-3.

TABLE 1-1
The number of collapsed animals in histamine evoked
asthma in conscious guinea-pigs after inhalation
of R-bambuterol or bambuterol aerosols (n = 8)

| Dose groups | 0 μg/Kg | 63 μg/Kg | 126 μg/Kg | 252 μg/Kg | 504 μg/Kg |
|---|---|---|---|---|---|
| R-bambuterol | (8)/8 | (7)/8 | (3)/8*^{▴} | (0)/8**^{▴▴} | (0)/8** |
| (Collapsed)/ | | | | | |
| No. in group | | | | | |
| RS-bambuterol | (8)/8 | (8)/8 | (7)/8 | (4)/8** | (0)/8** |
| (Collapsed)/ | | | | | |
| No. in group | | | | | |

*significant difference from control *p• •0.05• •**p• •0.01• •
^{▴}significant difference comparing to RS-BM. ^{▴}p• •0.05• •^{▴▴}p• •0.01

TABLE 1-2
The latency time in histamine evoked asthma in conscious guinea-pigs after
inhalation of R-bambuterol (R-BM) or bambuterol (RS-BM) aerosol (n = 8)

| Dose groups | 0 μg/Kg | 63 μg/Kg | 126 μg/Kg | 252 μg/Kg | 504 μg/Kg |
|---|---|---|---|---|---|
| R-bambuterol | 48 ± 9 | 95 ± 108^{▴▴} | 243 ± 161**^{▴} | 360 ± 0**^{▴} | 360 ± 0**^{▴▴} |
| Latency(sec.) | | | | | |
| RS-bambuterol | 48 ± 9 | 42 ± 9 | 85 ± 112 | 209 ± 162 | 360 ± 0** |
| Latency(sec.) | | | | | |

*Significant difference from control *p• •0.05, **p• •0.01.
^{▴}Significant difference comparing to RS-BM ^{▴}p• •0.05, ^{▴▴}p• •0.01.

Inhalation of either R-bambuterol or bambuterol aerosols showed a significant anti-asthma effects. However, the R-bambuterol is about twice potent as bambuterol, indicating that the R-bambuterol is the active enantiomer in racemic bambuterol.

### EXAMPLE 2

Comparison of the anti-asthma effects of R-bambuterol (R-BM) and bambuterol (RS-BM) administered by oral and by inhalation in conscious guinea pigs. Test methods: same as Example 1.

Study of dose-response: Same as Example 1, except the following: four hours before the exposure to aerosol histamine, animals were randomized into groups (n=8, equal male and female), and given R-bambuterol hydrochloride or racemic bambuterol hydrochloride in doses of 1.0, 2.0, 4.0 and 8.0 mg/kg, and vehicle control, orally via a stomach tube.

TABLE 2-1
The number of collapsed animals during histamine evoked
asthma in conscious guinea-pigs after oral administration
of R-bambuterol (R-BM) or bambuterol (RS-BM) (n = 8)

| | Dose groups (mg/Kg) | | | | |
|---|---|---|---|---|---|
| | 0 | 1.0 | 2.0 | 4.0 | 8.0 |
| R-BM | (8)/8 | (5)/8* | (2)/8**^{▴▴} | 0/8**^{▴▴} | (0)/8** |
| (Collapsed)/ | | | | | |
| No. in group | | | | | |
| RS-BM | (8)/8 | (6/)8* | (4)/8* | (2)/8** | (0)/8** |
| (Collapsed)/ | | | | | |
| No. in group | | | | | |

*Significant difference from control *p• •0.05• •**p• •0.01• •
^{▴}Significant difference comparing to RS-BM ^{▴}p• •0.05• •^{▴▴}p• •0.01

TABLE 2-2
The latency time in histamine evoked asthma in conscious guinea-pigs
after oral administration of R-bambuterol or bambuterol (n = 8)

| | Dose group (mg/Kg) | | | | |
|---|---|---|---|---|---|
| | 0 | 1.0 | 2.0 | 4.0 | 8.0 |
| R-BM | 43 ± 13• | 179 ± 151** | 295 ± 121**^{▴▴} | 360 ± 0**^{▴▴} | 360 ± 0** |
| Latency• •sec.• | | | | | |
| RS-BM1 | 44 ± 13• | 151 ± 130 | 227 ± 143** | 299 ± 113** | 360 ± 0** |
| Latency• •sec.• | | | | | |

*Significant difference from control *p• •0.05• •**p• •0.01• •
^{▴}Significant difference comparing to RS-BM ^{▴}p• •0.05• •^{▴▴}p• •0.01

These results show that doses required for maxunm anti-asthmatic effects (i.e. none of the histamine challenged animal in group are collapsed) were 4 mg/kg for R-bambuterol and 8 mg/kg for bambuterol. These doses are much greater than the doses required by inhalation (table 1-1;1-2). See table 2-3.

TABLE 2-3
Comparision of the maximum effective dose of R-bambuterol
and bambuterol required by oral and by inhalation.

| R-BM dose | Animal | RS-BM dose | Animal |
|---|---|---|---|
| (mg/Kg) | collapsed• •%• • | (mg/Kg) | collapsed• •%• • |
| Oral (4.0) | 0• •0/8• • | Oral (8.0 ) | 0• •0/8• • |
| Inhale (0.252) | 0• •0/8• • | Inhale (0.505) | 0• •0/8• • |
| Oral:Inhale = | | Oral:Inhale = | |
| 16:1 | | 16:1 | |

The results show that the maximum effective dose required by oral is 16 times higher than the dose required by inhalation. The much lower dose of R-bamubuterol or bambuterol required by inhalation will greatly reduce systemic adverse effects associated with the medicament.

### EXAMPLE 3

The time course of the anti-asthma effects of R-bambuterol (R-BM) and bambuterol (RS-BM) after administered by oral and by inhalation. Test Methods: same as example 1 except the following:

For orally administration, R-Bambuterol hydrochloride or racemic bambuterol hydrochloride at 4 and 8 mg/kg, and vehicle alone, were administered to the guinea pigs orally via a stomach tube. Exposure of treated animals to aerosol histamine (as above) was done at 1, 4 and 12 hours after R-bambuterol or bambuterol treatment. For each experimental group, there were a total of 8 animals with equal number in sex, and no repeated exposure was made for individual experimental animals.

For inhalation, R-Bambuterol hydrochloride or racemic bambuterol hydrochloride at 0.252 and 0.504 mg/kg, and vehicle alone, were nebulized and administered to the guinea pigs by inhalation. Exposure of drug treated animals to aerosol histamine (as above) was done at 1,4 and 12 hours after drug treatment. For each experimental group, there were a total of 8 animals with equal number in sex, and no repeated exposure was made for individual experimental animals.

Test Results Tables 3-1 and 3-2.

TABLE 3-1
The time course of anti-asthma effects by oral administration
of R-BM or RS-BM

| Drugs/ | Latency(sec) and (No. of collapsed) (n = 8) | | | |
|---|---|---|---|---|
| dose • mg/kg • • | Control | 60 min after | 240 min after | 720 min after |
| R-BM | 43 ± 13* (8)/8)* | 258 ± 141 (3/8) | 360 ± 0 (0/8) | 285 ± 129 (2/8) |
| 4.0 mg | | | | |
| RS-BM | 44 ± 13* (8/8)* | 260 ± 131 (3/8) | 360 ± 0 (0/8) | 360 ± 0 (0/8) |
| 8.0 mg | | | | |

*significant difference comparing to drug treatment <0.01 • •

TABLE 3-2
The time course of anti-asthmatic effects by inhalation of R-BM or RS-BM

| Drugs/dose | | Latency(sec) and (No. of collapsed) (n = 8) | | |
|---|---|---|---|---|
| • •μg/kg• • | Control | 60 min after | 240 min after | 720 min after |
| R-BM 252 μg | 48 ± 9* (8/8)* | 360 ± 0 (0/8) | 360 ± 0 (0/8) | 330 ± 84 (1/8) |
| RS-BM 504 μg | 48 ± 13* (8/8)* | 360 ± 0 (0/8) | 360 ± 0 (0/8) | 287 ± 136 (2/8• • |

*significant difference comparing to drug treatment (*p < 0.01• •

The results indicate that the time of maximum effect (Tmax) of R-bambuterol or bambuterol arrived within 240 min after oral, while the Tmax is only 60 min after inhalation. It is about four hours apart. Although its onset earlier, there is no earlier decline of the maximum anti-asthma effects in inhaled group in comparison of oral adminstraion, The anti-asthma effects are similar for both inhaled and oral group within 720 min. This similarity of effects can be even seen up to 24 hours for both groups. Therefore, within the duration of 12 hours or longer, the afficacy of R-bambuterol and bambuterol was higher when given by inhalation, its provide better protection against asthmatic attack in comparing oral administraion. This advantage was unexpected from prior arts.

### EXAMPLE 4

Effects of Inhalation of R-bambuterol or S-Bambuterol on Airway Resistance and Dynamic Pulmonary Compliance

Sensitization of animals: Ovalbumin (10 μg/ml) was injected into guinea-pigs subcutaneously and coincidentally with an intraperitoneal injection of B. pertussis vaccine. Identical injection of ovalbumin and adjuvant were made day 15 and 21 days later. On day 28th the animals were examined and used for studies.

Test Methods: The sensitized guinea pigs wer anaethetized. A cannula was inserted into the trachea. A plueral cannula was inserted into pleural space through the chest wall. Airway resistance (Raw) and dynamic pulmonary compliance (Cdyn) were measured by using the signals of intraplueral pressure, airflow and tidal volume.

The animals were restreined in closed chamber and nebulized R-bambuterol or S-bambuterol were inhaled at the dose of 126 μg/kg. After three minutes conditioning, the animals were then challenged with nebulized OVA to evoke asthma. Airway resistance (Raw) and dynamic pulmonary compliance (Cdyn) were measured before and after evoking asthma. Test results: results summarized in Table 4-1.

TABLE 4-1
Effects of Inhalation of R-Bambuterol (R-BM) aerosol
on the pulmonary functions in sensitized guinea
pigs• •n = 8• •

| | R-Bambuterol: 504 μg/kg | | |
|---|---|---|---|
| Dose Inhaled | Control | R-BM Inhaled | OVA Challenge |
| Raw | 10.73 ± 0.23 | 10.91 ± 0.41 | 11.51 ± 1.09 |
| Cdyn | 0.65 ± 0.01 | 0.70 ± 0.03 | 0.62 ± 0.02 |

In sensitized guinea pigs, Inhalation of R-Bambuterol results no significant changes both in Raw and Cdyn before OVA challenge. There is neither significant increase in Raw nor significant decrease in Cdyn from control value during OVA challenge, indicating a full protection of R-BM against the asthma attack. The pretreatment of nebulized R-bambuterol by inhalation and the OVA challenge is only about 3 minutes apart, indicating a fast onset of action by the inhaled drug.

However, when S-Bambuterol was given by inhalation, there was significant increase in Raw and decrease in Cdyn, indicating an airway constriction or spasm. In one incidence, inhale of S-bambuterol per se induced a collapes of a guinea pig.

Inhalation of S-bambuterol can produce a significant increase in airway resistance (Raw) and decrease in pulmonary compliance (Cdyn) at resting state before OVA challenge, indicating a worsening of pulmonary function. In one incidence, inhale of S-bambuterol per se induced a collapes of a guinea There was also a greater increase in airway resistance (Raw) and decrease in pulmonary compliance (Cdyn) during the asthma attack in animals treated with S-bambuterol by inhalation comparing to control.

The results show that S-bambuterol is inactive in protectoin against asthma attack and that using S-bambuterol per se could worsen the pulmonary function, which may be linked to the excerbation of asthma or hyper-responsiveness related to use of β2 agonists seen in clinic. Since racemic bambuterol consist of half amount of S-bambuterol. Therefore, using optic pure R-bambuterol as inhaled aerosols for treating respiratory disorders is a much safter alternative than racemic bambuterol.

### EXAMPLE 5

Enhancemnet of airway resistance in astham attack induced by oral administration of S-bambuterol.

Sensitized animals were divided into Saline (control), S-Bambuterol and R-bambuterol groups. R or S bambuterol were administered by both inhalation by oral (4 m/kg). Airway resistance (Raw) and dynamic pulmonary compliance (Cdyn) were measured at resting or during astham challenge by OVA. Animal preparation and experimental methods were similar as described previously.

In resting state, There are little changes in either Raw and Cdyn after S-bambuterol were given orally, which is different than given by inhalation noted above. There were significant increases in Raw in both control and S-bambuterol treated groups during the asthmatic attack by OVA, while there was little changes in Raw in R-bambuterol treated groups. The maximum increases in Raw were around 4 or 5 minutes after OVA challenge. The changes in Raw were greater in S-bambuterol treated group than control (saline) group. (Table 5)

TABLE 5
Changes in Airway resistance (Raw) in response
to OVA challenge in sensitized giunea pigs treated
with saline, S-bambuterol and R-bambuterol

| | Pretreated with | Pretreated with | Pretreated with |
|---|---|---|---|
| Time after OVA | Saline | S-bambuterol | R-Bambuterol |
| challenge | (n = 8) | (n = 8) | (n = 8) |
| Raw at 4^{th} min | 1.79 ± 1.07 | 2.40 ± 0.96^{▴▴} | 0.14 ± 0.13** |
| Raw at 5^{th} min | 1.72 ± 1.20 | 2.41 ± 0.98^{▴▴} | 0.11 ± 0.08** |

^{▴▴}Significant comparing to control and R-bambuerol
**Significant comparing to control and S-bambuterol

This reveals that the S-bambuterol, the distimor, can significantly worsen the asthmatic response. This may be explain the risk of excerbation of asthma or hyperresponsiveness associated with use of racemic β2 agonist, which consist of half amount of S-enantiomer. Therefore, R-bambuterol, administered either by oral or by inhalation, is a better alternative for treatment of asthma than racemi bambuterol. This advantage of R-bambuterol can not be anticipated in prior arts.

### EXAMPLE 6

Drug Tolerance to R-Bambuterol Induced by Pretreatment of S-Bambuterol

Animal sensitization and experiment methods were similar as discribed above. Briefly, animals were divided into three groups. R-bambuterol (8 mg/kg) or S-bamburerol (8 mg/kg) or 0.9% NaCL (control) were given orally in equal volum to each group seperatedly once a day for 7 consecutive days in order to induce drug tolerances. The Protection effects of same dosage of R-bambuterol against asthma attack induced by OVA challenge were evaluated by the changes of Airway resistance (Raw) and dynamic pulmonary compliance (Cdyn). The results listed in the table 6 below.

TABLE 6
Comparision of the protection effects of R-bambuterol against
asthma induced by OVA challenge in sensitized giunea pigs

| | Changes in Raw | Changes in Cdyn |
|---|---|---|
| Groups | During asthma | During asthma |
| S-BM pretreated• •n = 8• • | +0.36 ± 0.23^{▴} | −0.25 ± 0.10^{▴▴} |
| R-BM pretreated• •n = 8• • | +0.13 ± 0.09 | −0.11 ± 0.08 |
| Saline pretreated (n = 8) | +0.09 ± 0.03^{• • • •} | −0.08 ± 0.06^{• • • •} |

^{▴}comparing to R-BM pretreated group; *P < 0.05• •**P < 0.01• •
^{• •}comparing to S-BM pretreated group^{• •} ^{•}P^{•} < 0.0

In control group, R-bambuterol can fully protect against asthma attack. There are little changes in Raw and Cdyn. In R-bambuterol pretreated group, the protection effects of R-bambuterol are less than control but no significant differences in term of changes in Raw and Cdyn. However, In S-bambuterol pretreated group, the protection effects of R-bambuterol were significantly reduced. There were much greater increase in Raw and decrease in Cdyn during OVA challenge. This indicates that a drug tolerance to R-bambuterol were induced by S-bambuterol. Therefore, S-bambuterol play important role in inducing drug tolerance to R-bambuterol.

### EXAMPLE 6

Formulation of R-Bambuterol (R-BM) and Bambuterol (RS-BM) Solutions for Nebulization

Solutoins for inhalation by nebulation may be formulated with an aqueous vehicle with the addition of agents such as acid or alkali, buffer salts, isotonicity adjusting agents or antimicrobials. General method of preparation includes: Calculate the quantity of the individual ingredients required, for the total amount to be prepared; Accurately weigh/measure each of the ingredients; Dissolve the solids in about ⅔ of the volume of vehicle for the preparation; Add the liquid ingredients and mix well and filter through a 0.2μ filter system into sterile containers; Packaged into small units for single use. Benzalkonium Chloride may be added as needed (at ratio of 1:750 Solutions). Nebulization with a nebulizer.

Formula of R-BM (or RS-BM) 0.5% Inhaled Solution

| | | |
|---|---|---|
| R-Bambuterol | 500 | mg |
| Citric Acid, Anhydrous | 100 | mg |
| Sodium Chloride | 800 | mg |
| Sterile Water for Inhalation | qs 100 | mL |

### EXAMPLE 7

Formulation of R-Bambuterol and Bambuterol as Metered Dose Inhalers (MDI)

The micronised R-bambuterol (R-BM) or bambuterol (RS-BM) are weighed into an aluminiun can, HFA134a (1,1,1,2-tetrafluoroetane) is then added from a vacuum flask and a metering valve is crimped into place. For suspension aerosols, R-bambuterol or bambuterol should be micronised so as to permit inhalaiton of substantially amounts into the lungs upon administration. The particale size of micrnised R-bambuterol or bambuterol should less than 20 um, and perferably in the range 1 to 10 microns, for example, 1 to 5 microns. R-bambuterol or bambuterol may be prepared as solusions of propelent together with co-solvent such as ethanol and other ingredients. The dose of per actuation for R-bambuterol is 20-250 μg; perferable dose is 60 or 120 μg; The dose of per actuation for bambuterol is 60-500 μg, perferable dose is 120 to 240 μg. The propellet by weight is around 60-99.99% it may be adjusted according to other ingredients added.

Formula of R-BM or RS-BM pressured metered dose inhaler

| R-BM | 60 μg × 200 actuation | |
|---|---|---|
| Ethanol | 4% | |
| sorbitan trioleate | 0.012 | g |
| HFA134a | 8.0 | g |
| RS-BM | 120 μg × 200 actuation | |
| Ethanol | 8% | |
| sorbitan trioleate | 0.024 | g |
| HFA134a | 8.0 | g |

### EXAMPLE 8

R-Bambuterol(R-BM) and Bambuterol(RS-BM) Dry Powder Inhaler

R-bambuterol or bambuterol are micronised and bulk blended with the lactose in suitable proportions. The blend is filled into hard gelatin capsules or cartridges or in foil blister packs to be administered by an inhaler such as a Rotahaler, Diskhaler, or or other availible devices. Per capsule contains R-bambuterol 150 μg or bambuterol 300 μg.

Formula of R-BM or RS-BM dry powder inhaler

| R-BM | 15 | mg |
|---|---|---|
| Lacose | 125 | mg |
| Filled into 100 capsules | | |
| RS-BM | 30 | mg |
| Lacose | 250 | mg |

Filled into 100 capsules

### EXAMPLE 9

Metered Dose Inhaler (MDI) for R-Bambuterol (or Bambuterol) and Budesonide

Formula of MDI for R-BM (or RS-BM) and budesonide

| | | |
|---|---|---|
| R-BM | 30 | μg × 200 |
| Budesonide | 100 | μg × 200 |
| FHA134a | 8 | g |
| RS-BM | 60 | μg × 200 |
| Budesonide | 100 | μg × 200 |
| FHA134a | 8 | g |

The micronised R-bambuterol (or bambuterol) and budesonide are weighed into an aluminiun can, 1,1,1,2-tetrafluoroetane (HFA134a) is then added from a vacuum flask and a metering valve is crimped into place. Inhalers may be in suspension aerosols, or sulutions with addition of co-solvent such as ethanol and other ingredients. Daily dose for budesonide is 50-200 μg children or 100-500 μg (adults). Daily dose for R-bambuterol is 02-2 mg; for Bambuterol is 0.04-4 mg. R-bambuterol per actuation is 20-250 μm perferable dose is 60 or 120 μm, bambuterol per actuation is 60-500 μm perferable dose is 120 μm or 240 μm. The propellet by weight is around 60-99.99% it may be adjusted according to other ingredients added.

### EXAMPLE 10

Dry Powder Inhalar for R-Bambuterol or Bambuterol and Budesonide

Formula of dry powder inhalar of
R-BM (or RS-BM) and budesonide

| | | |
|---|---|---|
| R-BM | 15 | μg |
| Budesonide | 200 | μg |
| Lactose | 20 | mg |
| RS-BM | 30 | μg |
| Budesonide | 200 | μg |
| Lactose | 20 | mg |

Mironised R-bambuterol (or bambuterol) and micronised budesonide are bulk blended with the lactose in suitable proportions. The blend is filled into hard gelatin capsules or cartridges or in foil blister packs to be administered by an inhaler such as a Rotahaler, Diskhaler, or other availible devices.

### CITATION LIST

### Patent literature

1, Tan W and J. Cheng, R-Bambuterol, its preparation and pharmaceutical uses, U.S. Pat. No. 7,495,028, 2009.
2, Olsson et al., Bronchospasmolytic carbamate derivatives, U.S. Pat. No. 4,419,364, 1983
3, Olsson et al., Carbamate interdedimates for bronchospasmolytics, U.S. Pat. No. 4,451,663, 1984.

### NON PATENT LITERATURE

1. Svensson, Mechanism of action of bambuterol: a βagonist prodrug with sustained lung affinity, AAS 34, New Drug for Asthma Therapy, p 71-76, ©Birkhauser Verlag Basel, 1991.
2. Gunn et al., Comparision of the efficacy, tolerability and patient acceptability of once-daily bambuterol tablets against twice-daily controlled release salbutamol in nocturnal asthma. Eur J. Clin Pharmacol 48, p 23, 1995
3. Olsson, O A and L A. Svensson, New lipophilic terbutaline ester prodrugs with long effect duration, Pharmaceutical Research, Vol 1, page 19, 1984.
4. Ryrfeldt, A., E. Nilsson, A. Tunek and L A Svenssion, Bambuteral: uptake and metabolism in guinea pig isolated lungs, Pharmaceutical Research, Vol 5, page 154, 1988
5. Tunex, A. E. Levin and L A. Svenssion, Hydrolysis of ³H-bambuterol, a carbamate prodrug of terbutaline, in blood from human and laboratory animals in vitro. Biochemical Pharmacology. Vol. 37, page. 3871, 1988.

1. Use of R-bambuterol and its salts as medicament of inhaled formulations administered by inhalation into bronchioles and lungs for treatment of human or animal disorders with improved therapeutic effects and reduced toxicity.

2. The said R-bambuterol according to claim 1 includes bambuterol in which the, active ingredient is R-bambuterol.

3. The said R-bambuterol according to claims 1 is substantially optic pure form and has an enantiomer excess value not less than 98%.

4. The said R-bambuterol according to claims 1 is in an optic pure form and has an enantiomer excess value not less than 90%, and S-bambuterol should be no more than 5% by weight.

5. The-said disorders according to claim 1 are respiratory disorders including asthma and COPD (chronic obstructive pulmonary disease).

6. The said disorders according to claim 1 are lipid disorders including hyperlipidemia, hyperglycemia and obesity.

7. The said disorder according to claim 1 is preterm labor.

8. The said medicament according to claim 1 containing, separately or together, (A) R-bambuterol or a pharmaceutical acceptable salt thereof and (B) corticosteroids, for simultaneous, sequential or separate administration in the treatment of asthma, COPD or other respiratory disorders, wherein said (A) or (B), or (A) and (B) are in inhalable formulations.

9. The said corticosteroids according to claim 8 are budesonide, ciclesonide, beclomethasone, mometasone, flunisolide, fluticasone propionate, triamcinolone acetonide and/or its physiological acceptable salts and/or solvate thereof.

10. The said medicament according to claim 1 containing, separately or together, (A) R-bambuterol or a pharmaceutical acceptable salt thereof and (B) anticholinergics for simultaneous, sequential or separate administration for the treatment of asthma, COPD or other respiratory disorders, wherein said (A) or (B), or (A) and (B) are in inhalable forms.

11. The said anticholinergics according to claim 10 are ipratropium bromide, tiotropium, trospium, oxitropium, daratropium, atropine, homatropine, tropicamide, scopolamine, lycopyrolate, oxybutynin, tolterodine, and/or their salts.

12. The said medicament according to claim 1 containing, separately or together, (A) R-bambuterol or a pharmaceutically acceptable salt thereof and (B) short-acting β₂ agonists and their salts thereof for simultaneous, sequential or separate administration in the treatment of asthma, COPD or other respiratory disorders, wherein said (A) or (B), or (A) and (B) are in inhalable forms.

13. The said short-acting β2 agonists according to claim 12 are terbutaline, fenoterol, salbutamol, orciprenaline, clenbuterol, clorprenaline, reproterol, bitolterol, rimiterol, their chiral eutimors and their salts thereof.

14. The said inhalable forms according to claims 1 is an inhalable aerosol comprising medicament or medicament in solution or dispersion in a propellant, or an inhalable nebulizable composition comprising a dispersion of medicament or medicament in an aqueous, organic or aqueous/organic medium, or is mironised dry powder blended with lactose and packaged into a capsule for inhalars.

15. The said propellants according to claim 14 are 1,1,1,2-tetrafluoroethane (HFA134a) and/or 1,1,1,2,3,3,3-heptafluoropropane (HFA227).

16. The reduced toxicity according to claim 1 is adverse effects associated with use of R-bambuterol

17. The reduced toxicity according to claim 1 are drug tolerance and exacerbation of asthma associated with use of bambuterol.

18. Use of R-bambuterol as medicaments administered by inhalation or oral for treatment of asthma, COPD or other respiratory disorders with reduced drug tolerance or reduced risk of asthma exacerbation associated with bambuterol.
